(19) United States
(12) Patent Application Publication
(10) Pub. No.: US 2025/0134979 A1
(43) Pub. Date: May 1, 2025

(54) DNA VACCINE AGAINST LEISHMANIASIS

(71) Applicant: INTERVET INC., Madison, NJ (US)

(72) Inventors: Luciana ARAMUNI GONCALVES, Montes Claros (BR); Luis Fernando VETTORATO, Paulinia (BR); Francisco BONOMI BARUFI, Jacarei (BR); Helena FACCIOLI LOPES, Ribeirão Preto (BR); Mayka RABELO HENRIQUES, Montes Claros (BR); Jeane FERREIRA LEAL DE FREITAS, Montes Claros (BR); Simone Cristina ROSA, São Paulo (BR)

(73) Assignee: INTERVET INC., Madison, NJ (US)

(21) Appl. No.: 18/723,251

(22) PCT Filed: Dec. 22, 2022
(86) PCT No.: PCT/EP2022/087392

§ 371 Date: Jun. 21, 2024

(30) Foreign Application Priority Data
Dec. 23, 2021 (EP) 21217516.0

(51) Int. Cl. A61K 39/008; A61K 39/00; C07K 14/44; C07K 14/47

(52) U.S. Cl. CPC A61K 39/008; C07K 14/44; C07K 14/47; A61K 2039/552; A61K 2039/55577; C07K 2319/40

(57) ABSTRACT

The present invention relates to the fields of veterinary medicine and virology and provides a vaccine against leishmaniasis. In particular, the invention relates to an isolated polynucleotide comprising (i) a first expression cassette comprising a nucleic acid encoding heat shock protein (hsp) 65, and (ii) a second expression cassette comprising a nucleic acid encoding LACK (Leishmania homologue of receptors for activated C kinase). In other embodiments, the invention relates to a DNA plasmid comprising the polynucleotide, a DNA vaccine for use in the treatment of leishmaniasis, and a method of immunizing a subject against an infection with leishmaniasis.

The present invention relates to the fields of veterinary medicine and parasitology and provides a vaccine against leishmaniasis. In particular, the invention relates to an isolated polynucleotide comprising (i) a first expression cassette comprising a nucleic acid encoding mycobacterial heat shock protein (hsp) 65, and (ii) a second expression cassette comprising a nucleic acid encoding LACK (Leishmania homologue of receptors for activated C kinase). In other embodiments, the invention relates to a DNA plasmid comprising the polynucleotide, a DNA vaccine for use in the prophylactic treatment of leishmaniasis, and a method of immunizing a subject against an infection with leishmaniasis.

### BACKGROUND OF THE INVENTION

Leishmaniosis is a disease caused by protozoan parasites of the genus Leishmania. It is transmitted through the bites of female phlebotomine sand flies. There are numerous species of sand flies, only a minority of which are competent vectors of Leishmania. Dogs with or without clinical signs are infectious to sand flies and may transmit Leishmania parasites.

More than 23 species of Leishmania have been described, most of which are zoonotic. The most important Leishmania parasite to affect domestic animals is L. infantum, also known as L. chagasi in Latin America. Dogs are the main reservoir host for human visceral leishmaniosis caused by L. infantum, and the disease is potentially fatal in dogs and people. Because the internal organs and skin of the dog are affected, the canine disease is termed viscerocutaneous or canine leishmaniosis. Cats, horses, and other mammals can be infected by L. infantum or other Leishmania species. L. braziliensis, the cause of tegumentary canine leishmaniosis, is widespread in regions of South America.

Canine leishmaniosis is a major zoonosis endemic in >89 countries. It is prevalent in Europe, Africa, Asia, and South and Central America and vertically transmitted from dog to dog in the USA. It is also of concern in nonendemic countries where imported disease constitutes a veterinary and public health problem. Canine leishmaniosis is a multisystemic disease with a highly variable spectrum of immune responses and clinical manifestations. In endemic areas, the prevalence of dogs carrying infection is much higher than those demonstrating clinical disease. Clinical disease is associated with a marked antibody response that does not confer protection. In fact, immune-mediated mechanisms are responsible for much of the pathology in canine leishmaniosis. In most symptomatic dogs, the first sign of disease due to infection with Leishmania appears about 2-4 months after the initial infection. Symptoms may include sores on the skin, peeling, ulcers, loss of weight, bald patches, conjunctivitis, blindness, nasal discharge, muscular atrophy, inflammation, swelling, and organ failure, including mild heart attacks.

Diagnosis and treatment of affected individuals can be particularly complex, hindering infection control in endemic areas. The main chemotherapeutic protocol used for treatment of canine leishmaniosis includes N-methylglucamine antimoniate. Treatment frequently does not provide sterilizing cure. Treated dogs can remain carriers of infection and may relapse. They may remain infectious to sand flies.

Methods to prevent canine leishmaniosis include the use of topical insecticides, prophylactic immunotherapy and vaccination. Four vaccines against canine leishmaniosis have been licensed since 2004, two in Brazil (Leishmune®, the production and marketing license of which was withdrawn in 2014, and Leish-Tec®) and two in Europe (CaniLeish® and LetiFend®). Leish-Tec® is a recombinant vaccine, based on the Leishmania A2 antigen, against canine visceral leishmaniasis (CVL) After several years of marketing, doubts remain regarding vaccine efficacy and effectiveness, potential infectiousness of vaccinated and infected animals or the interference of vaccine-induced antibodies in L. infantum serological diagnosis.

In consequence, prophylactic treatment by vaccination against Leishmaniosis is urgently needed.

Studies on a protective vaccine candidate for leishmaniasis have advanced in recent years and several vaccination methods and several antigens were tested. Various killed or attenuated parasite based first generation vaccines, second generation vaccines based on antigenic protein or recombinant protein, and third generation vaccines derived from antigen-encoding DNA plasmids including heterologous prime-boost Leishmania vaccine have been examined for control and prevention of leishmaniasis). It has been elucidated that CD4+ T as well as CD8+ T lymphocytes play an important role in conferring defense against and cure of visceral leishmaniasis. Immunization with naked DNA (DNA vaccination) is a new approach that promotes both CD4+ and CD8+ mediated responses and helps to induce a protective response against infection.

A number of DNA vaccines have already been approved for commercial use in both companion and food animals. The mechanisms of action make DNA vaccines attractive for control of leishmaniasis. In the past few years, studies focused on some antigens such as GP63, CP, TSA, GP64, LmSTI1, LeIf and P8, p4 and LACK (Jain K and Jain N K. J. Immun. Methods, 422 (2015). LACK (Leishmania homologue of receptors for activated C kinase) is a 36 kDa protein localized in cytosol and external surface of the membrane. It is expressed in both promastigote and amastigote forms of the parasite. Genes implicated in host-gene interaction of Leishmania species are identified and reported in: Peacock et al., Nat Genet., 2007.

Mycobacterial HSPs, primarily Hsp65 and Hsp70, are known to modulate both the innate and adaptive (cellular and humoral) aspects of the immune system. DNA constructs encoding mycobacterial antigens as 65-kDa heat shock protein (hsp65), have been shown to induce significant protective immunity. A DNA vaccine (DNAhsp65) containing the mycobacterial hsp65 was recently developed. It was demonstrated that DNAhsp65 presents broad immunotherapeutic properties against various diseases (Silva C L, Malardo T and Tahyra A S C, Front. Med. Technol. 2:603-690 (2020)).

### SUMMARY OF THE INVENTION

In the present invention, it has surprisingly been found that vaccination with the 65-kDa heat shock protein hsp65 and LACK (Leishmania homologue of receptors for activated C kinase) using a DNA vaccine including the hsp65 and LACK genes achieves an effective immune response and reduced parasite load. This finding is particularly surprising, since it was recently shown in the art that an immune response induced by the LACK antigen does not provide protection against experimental Leishmania infection (Coelho et al., Infection and Immunity, 71(7), 2003).

Thus, in a first aspect, the present invention provides an isolated polynucleotide comprising: (i) a first expression cassette comprising a nucleic acid encoding heat shock protein (hsp) 65, or a functional fragment thereof, and (ii) a second expression cassette comprising a nucleic acid encoding LACK (Leishmania homologue of receptors for activated C kinase), or an immunogenic fragment thereof.

In a second aspect, the invention provides A DNA plasmid comprising the polynucleotide as described herein.

In a third aspect, the invention provides a DNA vaccine comprising the polynucleotide as described herein.

In a fourth aspect, the DNA vaccine is used in the protection of a subject against an infection with leishmaniasis.

In a fifth aspect, there is provided a method of immunizing a subject against an infection with leishmaniasis, the method comprising administering to a subject an immunogenetically effective amount of the DNA vaccine as described herein.

### DESCRIPTION OF FIGURES

FIG. 1: DNAHSP-LACK: schematic representation of the plasmid DNA vector described in Example 1 containing the hsp65 and LACK genes.

FIG. 2: Experimental design for immunogenicity and efficacy evaluation of vaccines and combinations resuspended in saline, in murine model.

FIG. 3: Representative graphs from immunogenicity and efficacy results in Example 3. 3A—IFN-γ response, 3B—Parasite burden in liver by limiting dilution assay.

FIG. 4: Representative graphs for parasite load by qPCR in lymph node in Example 4: 4A—43 days after challenge, 4B—190 days after challenge, 4C—302 days after challenge.

FIG. 5: Representative graphs for parasite load by qPCR in bone marrow in Example 4: 5A—43 days after challenge, 5B—190 days after challenge, 5C—302 days after challenge.

### DEFINITION OF TERMS

The term “nucleic acid (sequence)” or “polynucleic acid (sequence)” includes an RNA or DNA sequence. It may be single or double stranded. It may, for example, be genomic, recombinant, mRNA or cDNA. In the present invention, the polynucleic acid is typically double stranded DNA.

The term “isolated” is to be interpreted as: isolated from its natural context, by deliberate action or human intervention; e.g. by an in vitro procedure for biochemical purification.

Typically, a “(poly)nucleic acid” or “(poly)nucleic acid molecule” or “gene” encoding a protein, here: the antigens hsp65 or LACK according to the invention, is an open reading frame (ORF), indicating that no undesired stop-codons are present that would prematurely terminate the translation into protein. For the invention the nucleic acid molecule typically encodes the complete amino acid sequence of hsp65 or LACK. Alternatively, the “nucleic acid” or “nucleic acid molecule” may encode only one or more parts, in particular epitope regions, of the hsp65 or LACK protein for the recombinant expression of one or more functional (hsp65) or immunogenic (LACK) fragments thereof.

For the present invention, the exact nucleotide sequence of a nucleic acid molecule according to the invention is not critical, provided the nucleotide sequence allows the expression of the desired amino acid sequence, here: the desired hsp65 and LACK proteins. However, as is well known in the art, different nucleic acids can encode the same protein due to the ‘degeneracy of the genetic code’.

For the present invention, a nucleic acid molecule can be a DNA or an RNA molecule, but typically is DNA. This depends on the source material used for its isolation, and on the intended use. Methods to isolate one or the other type of molecule from a variety of starting materials, and of methods to convert one type into the other are well known to the skilled person.

An “isolated (poly)nucleic acid” according to the invention can conveniently be manipulated in the context of a vector, such as a DNA plasmid, when it is in DNA form. To allow an isolated (poly)nucleic acid molecule according to the invention to actually express hsp65 and LACK according to the invention, it will require proper expression control signals and a suitable environment, which are part of an “expression cassette”, which is defined herein as a (poly)nucleic acid encompassing all elements necessary for recombinant gene expression, such as recombinant gene expression from a DNA plasmid for the production of a protein of interest (POI).

For example, a nucleic acid molecule needs to be operatively linked to an upstream promoter element and needs to contain a translation start at the beginning of the coding sequence and a translation stop at the end of the coding sequence. Hence, an expression cassette for the recombinant expression of the hsp65 and LACK proteins typically comprises at least the hsp65 or LACK gene, which is under the control of a functional promoter. In addition, translational enhancers can be included upstream and/or downstream of the coding region to increase expression levels and which may also be part of an expression cassette. Typically, the plasmids and vectors used in the context of a particular expression system will provide for such elements and enhancers. Also, the bio-molecular machinery for transcription and translation is typically provided by the cells of the vaccinated subjects. By modifying the various elements and enhancers, the expression of antigens according to the invention can be optimised in e.g. timing, level, and quality; all this is within the routine capabilities of the skilled person. Therefore, in a preferred embodiment, the isolated nucleic acid molecule according to the invention in addition comprises expression control signals.

A “translational enhancer” is a nucleotide sequence forming an element, which can promote translation and, thereby, increase protein production. Typically, a translational enhancer may be found in the 5′ and 3′ untranslated regions (UTRs) of mRNAs. In particular, nucleotides in the 5′-UTR immediately upstream of the initiating ATG codon of the gene of interest (GOI) may have a profound effect on the level of translation initiation.

An “expression vector” (syn. “expression construct”), is usually a plasmid, typically a DNA plasmid, designed for recombinant gene expression in cells. The vector is used to introduce a specific gene into a target cell, and can commandeer the cell's mechanism for protein synthesis to produce the protein of interest (POI) encoded by the gene. In order to express the recombinant gene to produce the POI, the expression vector typically comprises at least a promotor to drive the expression of the GOI and may further comprise one or more translational enhancers to increase the yield of the POI.

The term “vaccine” as used herein refers to a preparation which, when administered to a subject, induces or stimulates a protective immune response. A vaccine can render an organism immune to a particular disease. In the context of the present invention, the vaccine can be or can comprise a DNA plasmid, thus also referred to as “DNA vaccine”, which may further contain a suitable carrier and/or adjuvant as described herein.

“DNA vaccination” involves immunization with a DNA plasmid encoding an antigen of the pathogen or an immunomodulator, i.e. with a “DNA vaccine”.

A “DNA vaccine” is a type of vaccine that transfects a specific antigen-coding DNA sequence onto the cells of an immunized species. DNA vaccines work by injecting genetically engineered plasmid containing the DNA sequence encoding the antigen(s) against which an immune response is sought, so the cells directly produce the antigen, thus causing a protective immunological response. The GOI is inserted into the DNA plasmid, along with appropriate genetic elements such as eukaryotic promoters for transcriptional control, a polyadenylation signal sequence for stable and effective translation, and a bacterial origin of replication. The plasmid is transfected into host cells, for example, via direct injection, or injection with an appropriate delivery system. The GOI then undergoes transcription and translation by host cellular machinery, resulting in the production of an antigenic protein that can induce innate and adaptive (cellular and humoral) immune responses.

DNA vaccines have number of advantages over other vaccination technologies that are of particular interest to veterinary medicine. They have the potential to be less expensive than other commercial vaccines, as they can be produced in large quantities by bacteria and, typically do not require expensive facilities of a high biosafety level. They are temperature stable and safe to transport, which can be important for farms located in remote areas or for wildlife vaccines that need to remain in the open for a prolonged period of time. Different genes can be combined simultaneously, which allows for the development of vaccines against multiple strains of a pathogen and for combination approaches against multiple pathogens. In addition, because the relevant protein is produced and presented intracellularly, both a cellular and humoral immune response are induced, which gives a more efficient immune response when the animal encounters the natural infection at a later timepoint.

DNA vaccines can be administered via a number of routes and techniques, which can alter the immune response and distribution of the protein. Typically, the DNA vaccines are administered by subcutaneous or intramuscular injection.

Thus, in the context of the present invention, the term “DNA vaccine” refers a DNA sequence encoding the nucleic acid sequences of the immunomodulator hsp65 and the antigen LACK, which are recombinantly expressed in the vaccinated subject to induce an immune response.

To “protect an animal against an infection with Leishmania” generally means aiding in preventing, ameliorating or curing a pathogenic infection with Leishmania, or aiding in preventing, ameliorating or curing a disorder arising from that infection, for example to prevent or reduce one or more clinical signs resulting from a post treatment (i.e. post vaccination) infection with Leishmania. The typical clinical signs which can be reduced through vaccination in the present invention include one or more of sores on the skin, peeling, ulcers, loss of weight, bald patches, conjunctivitis, blindness, nasal discharge, muscular atrophy, inflammation, swelling, and organ failure, including mild heart attacks.

In particular, the vaccine of the invention is aided in achieving an antibody and cellular immune response in the subject, which results in reduced parasite load following infection with Leishmania and a reduction or prevention of clinical signs of the disease. In a preferred embodiment, the vaccine of the invention is used in the prevention or amelioration of an infection with Leishmania. In this embodiment, vaccination with the vaccine of the invention achieves a reduction of one or more of the clinical signs and a reduced parasite load following infection. Most preferably, vaccination achieves a prevention of Leishmania infection.

The term “prevention” or “preventing” is intended to refer to averting, delaying, impeding or hindering the Leishmania infection by a prophylactic treatment. The vaccine may, for example, prevent or reduce the likelihood of an infectious Leishmania entering the subject.

### DETAILED DESCRIPTION OF EMBODIMENTS

### Polynucleic Acid and DNA Plasmid

The polynucleotide (or, synonymously, nucleic acid) of the first aspect is typically in the form of a DNA plasmid, such as a circular DNA plasmid, also referred to in the following as DNA vector or, briefly, vector, or is part of a DNA plasmid. Thus, in a further aspect, the present invention provides a DNA plasmid comprising the polynucleotide as described herein.

The polynucleotide of the invention comprises at least two expression cassettes for the recombinant expression of a gene encoding the heat shock protein hsp65 of mycobacteria and the LACK protein of leishmania.

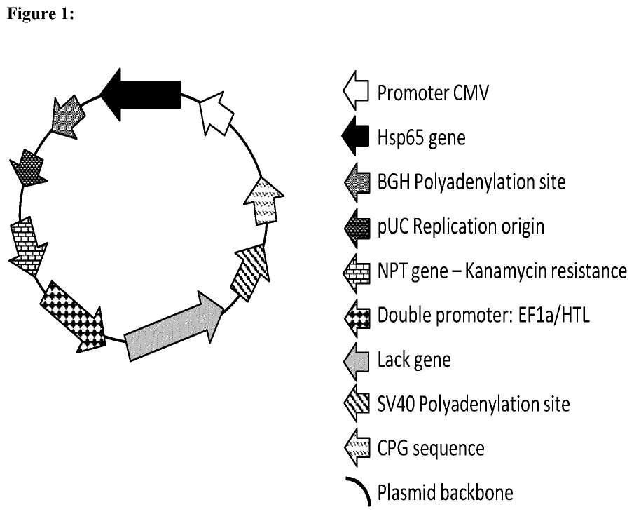

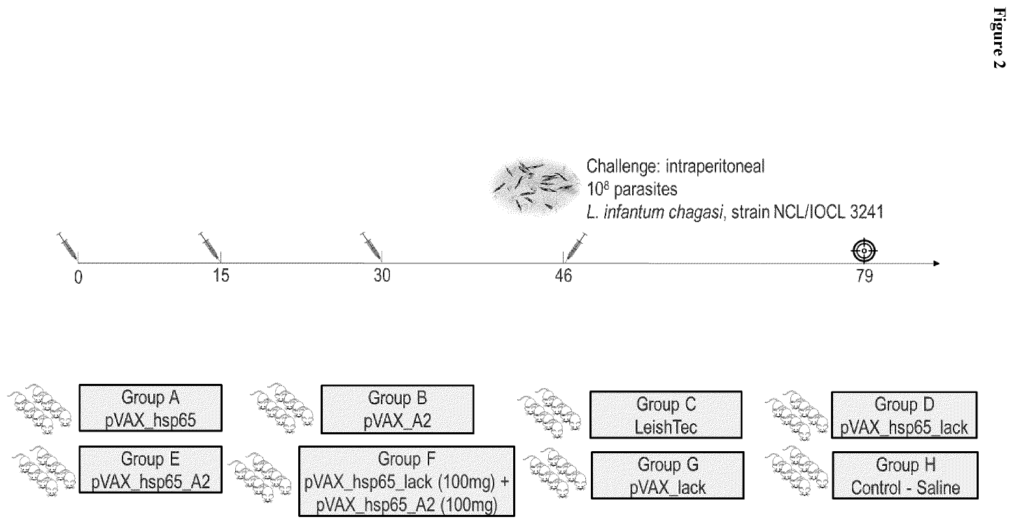

FIG. 1 shows an example of a construct of a DNA plasmid (DNAHSP-LACK) according to the invention, which comprises the two expression cassettes as described herein, i.e. one for the expression of the immunomodulator (hsp65) and one for the expression of the antigen (LACK).

The first expression cassette (i) comprises a nucleic acid encoding heat shock protein (hsp) 65, or an immunogenic fragment thereof.

Hsp65, as used herein, refers to the mycobacterial hsp65 gene that already showed ability to modulate and/or regulate the immune response in experimental model of several diseases with different Th1/Th2 pattern of immune response including leishmaniasis (Silva C L, Malardo T and Tahyra A S C, Front. Med. Technol. 2:603690 (2020)). Hence, in the polynucleotide construct of the present invention, the heat shock protein hsp65 of mycobacteria functions as an immunomodulator.

The amino acid sequence of hsp65 may be represented by the SEQ ID No. 1, referring to the amino acid sequence of hsp65 of mycobacterial strain Mycobacterium leprae, derived from the GenBank accession No.: AAA25354.1, which may be encoded by the nucleic acid sequence provided under GenBank Accession No. M14341.1 (M. leprae 65 kd antigen). Also encompassed by the present invention are functional fragments of the hsp65 protein containing the relevant parts of the protein essential to achieve an immunomodulatory effect. In particular, a functional fragment comprising the amino acids 48 to 583 of SEQ ID No. 1 is preferably used, since amino acids 1 to 47 of SEQ ID No. 1 are not related to the encoding and expression of the hsp65 protein.

Due to natural sequence variation between mycobacterial strains, the present invention also encompasses hsp65 amino acid sequences having at least 80% sequence identity to the amino acid sequence of SEQ ID NO. 1, or the functional fragments thereof, within conserved regions of the protein, preferably having at least 85% sequence identity to the amino acid sequence of SEQ ID NO. 1, most preferably having at least 90% sequence identity to the amino acid sequence of SEQ ID NO. 1, such as 91%, 92%, 93%, 94% or 95%, 97%, 98% or 99% sequence identity to the amino acid sequence of the Gene Bank Accession No. AAA25354.1, or functional fragments thereof.

The second expression cassette (ii) comprises a nucleic acid encoding LACK protein, or an immunogenic fragment thereof.

LACK (Leishmania homologue of receptors for activated C kinase), as referred to herein, is a 36 kDa protein, which is naturally located in the cytosol and on the outer membrane surface of Leishmania infantum and is expressed in both the promastigote and amastigote forms of the parasite. In the vaccine of the present invention, LACK is expressed from the polynucleotide of the invention and functions as the antigen for achieving an immune response against the LACK protein in the vaccinated subject. Hence, in the polynucleotide construct of the present invention, the LACK protein of leishmania functions as antigen to induce an immune response.

The amino acid sequence of LACK may be represented by the SEQ ID No. 2, referring to the amino acid sequence of the GenBank Accession No. ALP73427.1, which may be encoded by the nucleic acid sequence provided under GenBank Accession No. KT184317.1 (Leishmania infantum strain MHOM/TR/2009/EP174 activated protein kinase C receptor (LACK) gene). Also encompassed by the present invention are immunogenic fragments of the LACK protein containing the relevant epitopes to affect antigen binding and achieve an immunogenic response in the vaccinated subject.

Due to natural sequence variation between Leishmania strains, the present invention also encompasses LACK amino acid sequences having at least 80% sequence identity to the amino acid sequence of SEQ ID NO. 2, preferably having at least 85% sequence identity to the amino acid sequence of SEQ ID NO. 2, most preferably having at least 90% sequence identity to the amino acid sequence of SEQ ID NO. 2, such as 91%, 92%, 93%, 94% or 95%, 97%, 98% or 99% sequence identity to the amino acid sequence of the Gene Bank Accession No. ALP73427.1, or immunogenic fragments thereof.

Hsp65 and LACK may both be expressed under the control of the same or individual promoters, which are suitable for recombinant expression of the proteins in the vaccinated subject. Typically, hsp65 and LACK are expressed under the control of individual promoters Suitable promoters, which can be used for recombinant expression of hsp65 and LACK, are well-known in the art and described, for example, in: Poulain A. et al., J Biotechnol. 2017 255: 16-27; Kim S Y et al., J Biotechnol. 2002; 93(2):183-7; J. R. Deer, D. S. Allison, Biotech Progress 2004, 20(3): 880-889; R. V. Gopalkrishnan et al., Nucleic Acids Research, 1999 27(24): 4775-4782; Wang X. et al., J. Cell. Mol: Med. 2017 21(11): 3044-3054, the promoters described in these articles are incorporated herein by reference in their entirety.

For the construction of the vaccine of the present invention, a promoter named EF1α/HTLV can preferably be used. This promoter includes NotI and EcoRI restriction sites, which can be used for the insertion of genes for recombinant gene expression. EF1α-HTLV promoter is a composite promoter comprising the human Elongation Factor-1α (EF-1α) core promoter and the R segment and part of the U5 sequence (R-U5′) of the Human T-Cell Leukemia Virus (HTLV) Type 1 Long Terminal Repeat. The EF-1α promoter exhibits a strong activity and yields long lasting expression of a transgene in vivo. The R-U5′ has been coupled to the EF-1α core promoter to enhance stability of RNA. (Kim S Y et al., J Biotechnol. 2002; 93(2):183-7)

Thus, in a preferred embodiment, hsp65 is expressed under the control of the cytomegalovirus CMV promoter and LACK is expressed under the EF1α/HTLV promoter.

In addition, the expression cassette(s) may contain one or more elements used for regulating or enhancing the expression of the hsp65 and/or LACK, including expression enhancers or other cis-acting elements.

In a preferred embodiment, the expression cassette(s) contain one or more CpG motifs. CpG sequences in the plasmid backbone play an important adjuvant role in DNA vaccines. These sequences represent pairs of unmethylated CpG dinucleotides on the same DNA strand. For example, to increase the immunogenicity of the plasmid system, 1-10, such as 1, 2, 3, 4, 5, 6, 7, 8, 9, or 10, preferably 2-6, such as 3, 4, or 5, most preferably 4 CpG Motifs, can also added, and are typically present after the SV40 polyadenylation site of one or both expression cassette(s). In a particularly preferred embodiment, the expression cassette responsible for LACK expression comprises 4 CpG motifs.

In a further aspect, the polynucleotide or the DNA plasmid as described herein is used for the recombinant expression of hsp65 and LACK or immunogenic fragments thereof, which can be used as recombinant proteins in a vaccine for protection against Leishmania infection.

In a further aspect, the present invention provides a DNA construct, such as a DNA plasmid, containing the heat shock protein hsp65 gene of mycobacteria and the LACK gene of leishmania for use in the preparation of a pharmaceutical composition or medicament for the prevention of Leishmania infection.

In a further aspect, the present invention provides a DNA vaccine comprising the polynucleotide or the DNA plasmid as described herein.

### Carriers and Adjuvants

In a further aspect, the present invention provides a DNA vaccine as described herein, further comprising a pharmaceutically acceptable carrier and/or adjuvant.

Pharmaceutically acceptable carriers are well-known in the art. Merely as an example; such a carrier can be as simple as sterile water, saline or a buffer solution such as PBS. The vaccine may comprise a single carrier or a combination of two or more carriers. In a preferred embodiment, the pharmaceutically acceptable carrier is water or saline, and most preferably is saline.

Suitable carriers for the vaccine and use or method thereof according to the present invention and/or embodiments thereof are well known in the art and include diluent, adjuvant, antimicrobial agent, stabilizer, preservative, inactivating agent, or combination thereof. Suitably diluents may comprise distilled water, salts, medium ingredient, sterile water, buffer, stabilizer, preservative, bactericides, antibiotic, and chemicals to assist in dissolving.

The carrier may comprise, NaCl, sodium carbonate, thiomersal, buffered saline, sucrose, disodium adipate, disodium hydrogen phosphate, sodium-dihydrogen phosphate dihydrate.

The vaccine may contain pharmaceutically acceptable auxiliary substances such as pH adjusting and buffering agents, tonicity adjusting agents, wetting agents and the like, for example, sodium acetate, sodium lactate, sodium chloride, potassium chloride, calcium chloride, sorbitan monolaurate, triethanolamine oleate, and the like.

Suitable excipients in the vaccine according to the invention are hydrolysed gelatin, casein, sorbitol, and disodium phosphate dihydrate.

Stabilizers may be used e.g. to enhance the shelf-life of the vaccine, to improve freeze-drying efficiency, or to improve the appearance of the product. Useful stabilizers are i.a. SPGA, carbohydrates e.g. sorbitol, mannitol, trehalose, starch, sugars such as sucrose, dextran, lactose or glucose, amino acids such as glycine and monosodiumglutamate (salts of amino acids), proteins such as (recombinant) albumin, gelatin, hydrolysed collagen, or casein or degradation products thereof, and buffers, such as alkali metal phosphates.

In another aspect, the invention relates to a liquid vaccine composition of the present invention and a pharmaceutically acceptable carrier, wherein the carrier is a natural deep-eutectic solvent (NADES) preferably having a water activity of less than about 0.8. The NADES carrier is described in WO 2019/122329, which is hereby incorporated by reference in its entirety.

Further, the vaccine can contain an adjuvant, or may be free of an adjuvant (non-adjuvated vaccine). Adjuvants can be classified according to the source of their constituents, their physicochemical properties or their mechanism of action. Molecular adjuvants can act as immunostimulants (e.g., TLR ligands, cytokines, saponins and bacterial exotoxins that stimulate the immune response) and act directly on the immune system to enhance immune response against the antigen(s).

Since the vaccine of the invention includes hsp65 as immunostimulatory protein expressed from the polynucleotide of the invention, thus acting as an adjuvant, the addition of an (additional) adjuvant may be dispensable in the present invention. However, to affect an enhanced or more rapid immune response in the vaccinated subject, the addition of a further adjuvant may be desired.

Suitably adjuvants in vaccine compositions according to the invention and/or any embodiment thereof may include aluminum hydroxide, aluminum phosphate, saponins, saponins complexed with cholesterol, saponin complexed with cholesterol and lipids, non-metabolizable oil, mineral and/or plant/vegetable and/or animal oils, polymers, carbomers, surfactants, natural organic compounds, plant extracts, carbohydrates, cholesterol, lipids, water-in-oil emulsion, oil-in-water emulsion, water-in-oil-in-water emulsion, acylic acid polymer, acrylic acid saccharide cross-linked polymer, acrylic acid polyol cross-linked polymer.

Suitably adjuvants in vaccine compositions according to the invention and/or any embodiment thereof may include aluminum hydroxide, aluminum phosphate, saponins, Quil A, QS-21, GPI-0100, non-metabolizable oil, mineral and/or plant/vegetable and/or animal oils, polymers, carbomers, surfactants, natural organic compounds, plant extracts, carbohydrates, cholesterol, lipids, water-in-oil emulsion, oil-in-water emulsion, water-in-oil-in-water emulsion, HRA-3 (acrylic acid saccharide cross-linked polymer), HRA-3 with cottonseed oil (CSO), or HRA-5 (acrylic acid polyol cross-linked polymer), ISCOMATRIX. Suitably the adjuvant is a saponin, acrylic acid polymers, and/or aluminum hydroxide. Suitably saponins are Quil A, ISCOM, QS21, ISCOPREP 703, ISCOMATRIX, AS series, ISCOPREP saponin, GPIO100, AbISCO, Matrix M, Matrix C, Posintro. Suitably acrylic acid polymers are Carbopol such as HRA-3, HRA-5, Carbopol 940, Carbopol 941, Carbopol 934, Carbopol 971, Havlogen, CARBIGEN.

Suitable adjuvants are adjuvants based on saponin, more suitably adjuvants based on quillaia saponin. Examples of saponin based adjuvants are Quil A, ISCOM, QS21, ISCOMATRIX, ISCOPREP 703, AS series, GPI0100, AbISCO, Posintro. Suitable adjuvants are saponin based complexed with cholesterol. Suitable cholesterol complexed saponins are ASO1, AS15, AS02, ISCOM, ISCOMATRIX, Matrix-M, AbISCO.

Suitably, the adjuvant is at a concentration of about 0.01 to about 50%, or at a concentration of about 2% to 45%, or at a concentration of about 5% to about 40%, or at a concentration of about 7% to about 35%, or at a concentration of about 10% to about 30% by volume of the final product. Suitably, the adjuvant is at a concentration of about 0.01 to about 10%, or at a concentration of about 0.02 to about 5%, or at a concentration of about 0.05 to about 2.5%, or at a concentration of about 0.1 to about 2%, or at a concentration of about 0.15 to about 1.5%, or at a concentration of about 0.2 to about 1.2%, or at a concentration of about 0.3 to about 1%, or at a concentration of about 0.4 to about 0.8%, or at a concentration of about 0.5 to about 0.7%,

Therefore, preferably, the vaccine according to the invention is in a freeze-dried form, i.e. is provided as a lyophilizate.

The vaccine may also comprise, or be capable of expressing, another active agent, for example one which may stimulate early protection prior to the LACK-induced adaptive immune response. The active agent may also be another antigen capable of inducing an immune response in the vaccinated subject against another disease (combination vaccine).

### Vaccines and Production Thereof

In vitro recombinant DNA methods known to the skilled person can be used to generate a polynucleotide molecule according to the invention, comprising the two expression cassettes (i) and (ii) for the recombinant expression of hsp65 and LACK. Conveniently, this can be done by making and sub-cloning PCR fragments, or by de novo gene synthesis techniques.

Alternatively, a polynucleotide molecule according to the invention can be produced via an in vitro cell-based expression system, as this provides advantages in respect of yields and safety. The expression system can be based on prokaryotic or eukaryotic cells; if eucaryotic, can be based on host cells from a yeast, mammalian, insect, or plant, all as described in the prior art.

The present invention thus further relates to the production of the polynucleotide, such as a DNA plasmid, as described above, and which is used in the production of a vaccine. In particular, the DNA plasmid according to the invention may be used for vaccination of subjects. Preferably, the DNA plasmid is incorporated into a composition comprising the DNA plasmid and one or more pharmaceutically acceptable carrier(s) and/or adjuvant(s).

In a further aspect, the present invention provides a DNA vaccine for use in the protection of a subject against an infection with leishmaniasis. The subject is not particularly limited and can be animal or human. Preferably, the subject is dog, which is an animal species susceptible to infection with canine leishmaniasis. Thus, the preferred utility of the embodiments of the present invention is in veterinary medical use, in particular for vaccination of dogs against Leishmaniasis.

Infection can be caused by more than 20 of about 30 known Leishmania species that infect mammals. The vaccine of the invention may provide protection against one or more than one Leishmania species. In particular, since the LACK region is conserved among Leishmania species, the vaccine of the present invention can achieve broad protection against a wide range of Leishmania species. Thus, in a preferred embodiment, the vaccine of the present invention provides protection against infection with the following Leishmania species L. donovani complex with 3 species (L. donovani, L. infantum, and L. chagasi); the L. mexicana complex with 3 main species (L. mexicana, L. amazonensis, and L. venezuelensis); L. tropica; L. major; L. aethiopica; and the subgenus Viannia with 4 main species (L. (V.) braziliensis, L. (V.) guyanensis, L. (V.) panamensis, and L. (V.) peruviana). A majority of infections is caused by L. infantum. The different species are morphologically indistinguishable, but they can be differentiated by isoenzyme analysis, molecular methods, or monoclonal antibodies.

The vaccine may be used therapeutically, to treat an existing Leishmania infection, but preferably is used prophylactically, to block or reduce the likelihood of infection and/or to ameliorate the clinical signs of infection and/or to prevent or reduce the likelihood of spreading the disease.

The present invention also provides a method of protecting a subject against an infection with Leishmania by administration of an effective amount of a vaccine of the present invention. A method of protecting a subject against an infection with Leishmania comprises the step of producing a DNA plasmid as described above, optionally adding a pharmaceutically acceptable carrier and/or adjuvant and administering the vaccine to the subject.

Further, the present invention provides a polynucleotide as described herein for use in the manufacture of a DNA vaccine for protecting a subject against an infection with Leishmania,

### Administration

The present invention contemplates at least one administration to an animal of an efficient amount of the DNA vaccine according to the invention. A DNA vaccine can be administered in any art-known method, including any local or systemic method of administration. Administration can be performed e.g. by administering the antigens into muscle tissue (intramuscular, IM), into the dermis (intradermal, ID), underneath the skin (subcutaneous, SC), underneath the mucosa (submucosal, SM), in the veins (intravenous, IV), into the body cavity (intraperitoneal, IP), orally, anally etc. For the current vaccine IM, ID and SC administration are preferred.

The invention is directed to the following embodiments:

In a first embodiment, the invention is directed to an isolated polynucleotide comprising:
(i) a first expression cassette comprising a nucleic acid encoding heat shock protein (hsp) 65, or a functional fragment thereof, and
(ii) a second expression cassette comprising a nucleic acid encoding LACK (Leishmania homologue of receptors for activated C kinase), or an immunogenic fragment thereof.

In embodiment 2, the invention is directed to the polynucleotide of embodiment 1, wherein hsp 65 comprises the amino acids 48 to 583 of the amino acid sequence according to SEQ ID NO:1, or comprises an amino acid sequence having at least 90% sequence identity thereof.

In embodiment 3, the invention is directed to the polynucleotide of embodiment 1 or 2, wherein LACK comprises the amino acid sequence according to SEQ ID NO:2 or comprises an amino acid sequence having at least 90% sequence identity thereof.

In embodiment 4, the invention is directed to the polynucleotide of any one of the preceding embodiments, wherein the nucleic acids encoding hsp65 and LACK are under the control of different promoters.

In embodiment 5, the invention is directed to the polynucleotide of any one of the preceding embodiments, wherein the nucleic acid encoding hsp65 is under the control of the cytomegalovirus (CMV) promoter.

In embodiment 6, the invention is directed to the polynucleotide of any one of the preceding embodiments, wherein the nucleic acid encoding LACK is under the control of the EF1α-HTLV promoter.

In embodiment 7, the invention is directed to the polynucleotide of any one of the preceding embodiments, wherein the second expression cassette further comprises one or more CpG motifs.

In embodiment 8, the invention is directed to a DNA plasmid comprising the polynucleotide of any one of embodiments 1-7.

In embodiment 9, the invention is directed to the use of the polynucleotide of any one of embodiments 1 to 7 or of the DNA plasmid of embodiment 8 for the recombinant expression of hsp65 and LACK or immunogenic fragments thereof.

In embodiment 10, the invention is directed to a DNA vaccine comprising the polynucleotide of any one of embodiments 1 to 7 or comprising the DNA plasmid of embodiment 8.

In embodiment 11, the invention is directed to the DNA vaccine of embodiment 10, further comprising a pharmaceutically acceptable carrier.

In embodiment 12, the invention is directed to the DNA vaccine of embodiment 10 or 11, which is a non-adjuvated vaccine.

In embodiment 13, the invention is directed to the DNA vaccine of embodiment 10 or 11, which comprises an adjuvant.

In embodiment 14, the invention is directed to the DNA vaccine of embodiment 13, wherein the adjuvant is saponin.

In embodiment 15, the invention is directed to the DNA vaccine of any one of embodiments 10 to 14 for use in the protection of a subject against an infection with leishmaniasis.

In embodiment 16, the invention is directed to the DNA vaccine for use of embodiment 15, wherein the subject is dog.

In embodiment 17, the invention is directed to the DNA vaccine for use of embodiment 15 or 16, wherein the infection is with canine leishmaniasis.

In embodiment 18, the invention is directed to a method of immunizing a subject against an infection with leishmaniasis, the method comprising administering to a subject an immunogenetically effective amount of the DNA vaccine of any one of embodiments 10 to 14.

It should be understood that combinations of embodiments as described herein are envisioned and for brevity only a number of exemplifying combinations are explicitly described. However this should not be interpreted as limiting to the embodiments as described.

| The amino acid sequence SEQ ID No. 1: | |
|---|---|
| Met Pro Gly Arg Asp Gly Glu Thr Gln Pro Ala Ser Cys Gly Arg Pro | |
| 1 5 10 15 | |
| Ser Arg Ala Leu His Pro Ala Ser Val Ser Asn Gly Gly Cys Arg His | |
| 20 25 30 | |
| Pro Val Thr Leu Ala Ser Phe Leu Ile Arg Arg Asn His Phe Ala Met | |
| 35 40 45 | |
| Ala Lys Thr Ile Ala Tyr Asp Glu Glu Ala Arg Arg Gly Leu Glu Arg | |
| 50 55 60 | |
| Gly Leu Asn Ser Leu Ala Asp Ala Val Lys Val Thr Leu Gly Pro Lys | |
| 65 70 75 80 | |
| Gly Arg Asn Val Val Leu Glu Lys Lys Trp Gly Ala Pro Thr Ile Thr | |
| 85 90 95 | |
| Asn Asp Gly Val Ser Ile Ala Lys Glu Ile Glu Leu Glu Asp Pro Tyr | |
| 100 105 110 | |
| Glu Lys Ile Gly Ala Glu Leu Val Lys Glu Val Ala Lys Lys Thr Asp | |
| 115 120 125 | |
| Asp Val Ala Gly Asp Gly Thr Thr Thr Ala Thr Val Leu Ala Gln Ala | |
| 130 135 140 | |
| Leu Val Lys Glu Gly Leu Arg Asn Val Ala Ala Gly Ala Asn Pro Leu | |
| 145 150 155 160 | |
| Gly Leu Lys Arg Gly Ile Glu Lys Ala Val Asp Lys Val Thr Glu Thr | |
| 165 170 175 | |
| Leu Leu Lys Asp Ala Lys Glu Val Glu Thr Lys Glu Gln Ile Ala Ala | |
| 180 185 190 | |
| Thr Ala Ala Ile Ser Ala Gly Asp Gln Ser Ile Gly Asp Leu Ile Ala | |
| 195 200 205 | |
| Glu Ala Met Asp Lys Val Gly Asn Glu Gly Val Ile Thr Val Glu Glu | |
| 210 215 220 | |
| Ser Asn Thr Phe Gly Leu Gln Leu Glu Leu Thr Glu Gly Met Arg Phe | |
| 225 230 235 240 | |
| Asp Lys Gly Tyr Ile Ser Gly Tyr Phe Val Thr Asp Ala Glu Arg Gln | |
| 245 250 255 | |
| Glu Ala Val Leu Glu Glu Pro Tyr Ile Leu Leu Val Ser Ser Lys Val | |
| 260 265 270 | |
| Ser Thr Val Lys Asp Leu Leu Pro Leu Leu Glu Lys Val Ile Gln Ala | |
| 275 280 285 | |
| Gly Lys Ser Leu Leu Ile Ile Ala Glu Asp Val Glu Gly Glu Ala Leu | |
| 290 295 300 | |
| Ser Thr Leu Val Val Asn Lys Ile Arg Gly Thr Phe Lys Ser Val Ala | |
| 305 310 315 320 | |
| Val Lys Ala Pro Gly Phe Gly Asp Arg Arg Lys Ala Met Leu Gln Asp | |
| 325 330 335 | |
| Met Ala Ile Leu Thr Gly Ala Gln Val Ile Ser Glu Glu Val Gly Leu | |
| 340 345 350 | |
| Thr Leu Glu Asn Thr Asp Leu Ser Leu Leu Gly Lys Ala Arg Lys Val | |
| 355 360 365 | |
| Val Met Thr Lys Asp Glu Thr Thr Ile Val Glu Gly Ala Gly Asp Thr | |
| 370 375 380 | |
| Asp Ala Ile Ala Gly Arg Val Ala Gln Ile Arg Thr Glu Ile Glu Asn | |
| 385 390 395 400 | |
| Ser Asp Ser Asp Tyr Asp Arg Glu Lys Leu Gln Glu Arg Leu Ala Lys | |
| 405 410 415 | |
| Leu Ala Gly Gly Val Ala Val Ile Lys Ala Gly Ala Ala Thr Glu Val | |
| 420 425 430 | |
| Glu Leu Lys Glu Arg Lys His Arg Ile Glu Asp Ala Val Arg Asn Ala | |
| 435 440 445 | |
| Lys Ala Ala Val Glu Glu Gly Ile Val Ala Gly Gly Gly Val Thr Leu | |
| 450 455 460 | |
| Leu Gln Ala Ala Pro Ala Leu Asp Lys Leu Lys Leu Thr Gly Asp Glu | |
| 465 470 475 480 | |
| Ala Thr Gly Ala Asn Ile Val Lys Val Ala Leu Glu Ala Pro Leu Lys | |
| 485 490 495 | |
| Gln Ile Ala Phe Asn Ser Gly Met Glu Pro Gly Val Val Ala Glu Lys | |
| 500 505 510 | |
| Val Arg Asn Leu Ser Val Gly His Gly Leu Asn Ala Ala Thr Gly Glu | |
| 515 520 525 | |
| Tyr Glu Asp Leu Leu Lys Ala Gly Val Ala Asp Pro Val Lys Val Thr | |
| 530 535 540 | |
| Arg Ser Ala Leu Gln Asn Ala Ala Ser Ile Ala Gly Leu Phe Leu Thr | |
| 545 550 555 560 | |
| Thr Glu Ala Val Val Ala Asp Lys Pro Glu Lys Thr Ala Ala Pro Ala | |
| 565 570 575 | |
| Ser Asp Pro Thr Gly Gly Met Gly Gly Met Asp Phe | |
| 580 585 | |
| The amino acid sequence SEQ ID No. 2: | |
| Met Asn Tyr Glu Gly His Leu Lys Gly His Arg Gly Trp Val Thr Ser | |
| 1 5 10 15 | |
| Leu Ala Cys Pro Gln Gln Ala Gly Ser Tyr Ile Lys Val Val Ser Thr | |
| 20 25 30 | |
| Ser Arg Asp Gly Thr Ala Ile Ser Trp Lys Ala Asn Pro Asp Arg His | |
| 35 40 45 | |
| Ser Val Asp Ser Asp Tyr Gly Leu Pro Ser His Arg Leu Glu Gly His | |
| 50 55 60 | |
| Thr Gly Phe Val Ser Cys Val Ser Leu Ala His Ala Thr Asp Tyr Ala | |
| 65 70 75 80 | |
| Leu Thr Ala Ser Trp Asp Arg Ser Ile Arg Met Trp Asp Leu Arg Asn | |
| 85 90 95 | |
| Gly Gln Cys Gln Arg Lys Phe Leu Lys His Thr Lys Asp Val Leu Ala | |
| 100 105 110 | |
| Val Ala Phe Ser Pro Asp Asp Arg Leu Ile Val Ser Ala Gly Arg Asp | |
| 115 120 125 | |
| Asn Val Ile Arg Val Trp Asn Val Ala Gly Glu Cys Met His Glu Phe | |
| 130 135 140 | |
| Leu Arg Asp Gly His Glu Asp Trp Val Ser Ser Ile Cys Phe Ser Pro | |
| 145 150 155 160 | |
| Ser Leu Glu His Pro Ile Val Val Ser Gly Ser Trp Asp Asn Thr Ile | |
| 165 170 175 | |
| Lys Val Trp Asn Val Asn Gly Gly Lys Cys Glu Arg Thr Leu Lys Gly | |
| 180 185 190 | |
| His Ser Asn Tyr Val Ser Thr Val Thr Val Ser Pro Asp Gly Ser Leu | |
| 195 200 205 | |
| Cys Ala Ser Gly Gly Lys Asp Gly Ala Ala Leu Leu Trp Asp Leu Ser | |
| 210 215 220 | |
| Thr Gly Glu Gln Leu Phe Lys Ile Asn Val Glu Ser Pro Ile Asn Gln | |
| 225 230 235 240 | |
| Ile Ala Phe Ser Pro Asn Arg Phe Trp Met Cys Val Ala Thr Glu Arg | |
| 245 250 255 | |
| Ser Leu Ser Val Tyr Asp Leu Glu Ser Lys Ala Val Ile Ala Glu Leu | |
| 260 265 270 | |
| Thr Pro Asp Gly Ala Lys Pro Ser Glu Cys Ile Ser Ile Ala Trp Ser | |
| 275 280 285 | |
| Ala Asp Gly Asn Thr Leu Tyr Ser Gly His Lys Asp Asn Leu Ile Arg | |
| 290 295 300 | |
| Val Trp Ser Ile Ser Asp Ala Glu | |
| 305 310 | |
| Hsp65_Lack Expression Construct SEQ ID No. 3: | |
| gactcttcgc gatgtacggg ccagatatac gcgttgacat tgattattga ctagttatta | 60 |
| atagtaatca attacggggt cattagttca tagcccatat atggagttcc gcgttacata | 120 |
| acttacggta aatggcccgc ctggctgacc gcccaacgac ccccgcccat tgacgtcaat | 180 |
| aatgacgtat gttcccatag taacgccaat agggactttc cattgacgtc aatgggtgga | 240 |
| ctatttacgg taaactgccc acttggcagt acatcaagtg tatcatatgc caagtacgcc | 300 |
| ccctattgac gtcaatgacg gtaaatggcc cgcctggcat tatgcccagt acatgacctt | 360 |
| atgggacttt cctacttggc agtacatcta cgtattagtc atcgctatta ccatggtgat | 420 |
| gcggttttgg cagtacatca atgggcgtgg atagcggttt gactcacggg gatttccaag | 480 |
| tctccacccc attgacgtca atgggagttt gttttggcac caaaatcaac gggactttcc | 540 |
| aaaatgtcgt aacaactccg ccccattgac gcaaatgggc ggtaggcgtg tacggtggga | 600 |
| gctctatata agcagagctc tctggctact agagaaccca ctgcttactc gcttatcgaa | 660 |
| attaatacga ctcactatag ggagacccaa gctggctagc gtttaaactt aagcttggta | 720 |
| ccgagctcgg atccgatact tcgcaatggc caagacaatt gcgtacgacg aagaggcccg | 780 |
| tcgcggcctc gagcggggct tgaacagcct cgccgacgcg gtaaaggtga cgttgggtcc | 840 |
| gaaggggcgc aacgtcgttc tagagaagaa gtggggtgct cccacgatca ccaacgatcg | 900 |
| cgtgtccatc gccaaggaga tcgagctgga ggacccgtac gagaagattg gcgctgagtt | 960 |
| ggtcaaggaa gtcgccaaga agacagatga cgtcgccggt gatggcacca cgacggccac | 1020 |
| cgtgctggcc caggcattgg tcaaagaggg cctacgcaac gtcgcggccg gcgccaaccc | 1080 |
| gctaggtctc aagcgtggca tcgagaaagc tgtcgataag gtaactgaga ctctgctcaa | 1140 |
| ggacgctaag gaggtcgaaa ccaaggaaca aattgctgcc actgcagcga tttcggccgg | 1200 |
| tgaccagtcg atcggtgatc tgatcgccga ggcgatggac aaggttggca acgagggtgt | 1260 |
| tatcaccgtc gaggaatcca acaccttcgg tctgcagctc gagctcaccg agggaatgcg | 1320 |
| gttcgacaag ggctacattt cgggctactt cgtcaccgac gccgagcgtc aggaagctgt | 1380 |
| cctagaggag ccctacatcc ttctggtcag ctccaaagtg tctaccgtca aggacctgct | 1440 |
| gccgctgcta gagaaggtca tccaggccgg caagtcgctg ctgatcattg ctgaggatgt | 1500 |
| cgagggtgag gcgttgtcta ccctggtcgt caacaagatc cgtggcactt tcaagtcggt | 1560 |
| ggcggtcaaa gctcctggct ttggtgaccg ccgcaaggca atgttgcaag acatggccat | 1620 |
| tctcaccgga gcccaggtca tcagcgagga ggtcggtctc acattggaga acaccgatct | 1680 |
| gtcattgctg ggcaaggccc gcaaggtggt tatgaccaag gacgaaacca ccatcgtcga | 1740 |
| gggtgccggt gacaccgacg ccatcgccgg gcgagtcgct cagatccgta ccgagatcga | 1800 |
| gaacagtgac tctgactatg accgcgagaa actgcaggaa cgcctggcta agttggccgg | 1860 |
| tggtgttgcg gtgatcaagg ccggtcctgc cactgaggtg gagctcaagg agcgcaagca | 1920 |
| ccgcatcgag gacgcagtcc gcaacgccaa ggccgccgtg gaggagggga tcgtcgccgg | 1980 |
| ccgcggtgtg actctgctac aggctgctcc ggctctggac aagctgaagc tgaccggtga | 2040 |
| cgaggcgacc ggtgccaata ttgtcaaggt ggcgttcgaa gctccgctca agcagatcgc | 2100 |
| cttcaattcc gggatggagc ccggcgtcgt ggccgaaaag gtgcgtaacc tttcagtggg | 2160 |
| tcacggcctg aacgccgcca ccggtgagta cgaggacctg ctcaaggccg gcgttgccga | 2220 |
| cccggtgaag gttacacgtt ctgcgctgca gaacgcagcg tccatcgccg gcctgttcct | 2280 |
| tactacggag gccgtcgtcg ccgacaagcc ggagaagacg gcagctccgg cgagcgaccc | 2340 |
| gaccggtggc atggtgacgt ccggtcatga tgcaggtagc tacgtggtct gagggcccgt | 2400 |
| ttaaacccgc tgatcagcct cgactgtgcc ttctagttgc cagccatctg ttctttgccc | 2460 |
| ctcccccgtg ccttccttga ccctggaagg tgccactccc actgtccttt cctaataaaa | 2520 |
| tgaggaaatt gcatcgcatt gtctgagtag gtgtcattct attctggggg gtggggtggg | 2580 |
| gcaggacagc aagggggagg attcggaaga caatagcagg catgctgggg atgcggtggg | 2640 |
| ctctatggct tctactgggc ggttttatgg acagcaagcg aaccggaatt gccagctcgg | 2700 |
| gcgccctctg gtaaggttgg gaagccctgc aaagtaaact ggatggcttt ctcgccgcca | 2760 |
| aggatctgat ggcgcagggg atcaagctct gatcaagaga caggatgagg atcgtttcgc | 2820 |
| atgattgaac aagatggatt gcacgcaggt tctccggccg cttgggtgga gaggctattc | 2880 |
| ggctatgact gggcacaaca gacaatcggc tgctctgatg ccgccgtgtt ccggctgtca | 2940 |
| gcgcaggggc gcccggttct ttttgtcaag accgacctgt ccggtgccct gaatgaactg | 3000 |
| caagacgagg cagcgcggct atcgtggctg gccacgacgg gcgttccttg cgcagctgtg | 3060 |
| ctcgacgttg tcactgaagc gcgaagggac tggctgctat tgcgcgaagt gccggggcag | 3120 |
| gatctcctgt catctcacct tgctcctgcc gagaaagtat ccatcatggc tgatgcaatg | 3180 |
| cggcggctgc atacgcttga tccggctacc tgcccattcg accaccaagc gaaacatcgc | 3240 |
| atcgagcgag cacgtactcg gatggaagcc ggtcttgtcg atcaggatga tctggacgaa | 3300 |
| gagcatcagg ggctcgcgcc agccgaactg ttcgccaggc tcaaggcgag catgcccgac | 3360 |
| ggcgaggatc tcgtcgtgac ccatggcgat gcctgcttgc cgaatatcat ggtggaaaat | 3420 |
| ggccgctttt ctggattcat cgactgtcgc cggctgggtg tggcggaccg ctatcaggac | 3480 |
| atagcgttgg ctacccgtga tattgctgaa gagcttggcg gcgaatgggc tgaccgcttc | 3540 |
| ctcgtgcttt acggtatcgc cgctcccgat tcgcagcgca tcgccttcta tcgccttctt | 3600 |
| gacgagttct tctgaattat taacgcttac aatttcctga tccggtattt tctccttacg | 3660 |
| catctgtgcg gtatttcaca ccgcatacag gtcgcacttt tcggggaaat gtgcgcggaa | 3720 |
| cccctatttg tttatttttc taaatacatt caaatatgta tccgctcatg agacaataac | 3780 |
| cctgataaat gcttcaataa tagcaccccg ggatctgcga tcgctccggt gcccgtcagt | 3840 |
| gggcagagcg cacatcgccc acagtccccg agaagttggg gggaggggtc ggcaattgaa | 3900 |
| cgggtgccta gagaaggtgg cgcggggtaa actgggaaag tgatgtcgtg tactggctcc | 3960 |
| gcctttttcc cgagggtggg ggagaaccgt atataagtgc agtagtcgcc gtgaacgttc | 4020 |
| tttttcgcaa cgggtttgcc gccagaacac agctgaagct tcgaggggct cgcatctctc | 4080 |
| cttcacgcgc ccgccgccct acctgaggcc gccatccacg ccggttgagt cgcgttctgc | 4140 |
| cgcctcccgc ctgtggtgcc tcctgaactg cgtccgccgt ctaggtaagt ttaaagctca | 4200 |
| ggtcgagacc gggcctttgt ccggcgctcc cttggagcct acctagactc agccggctct | 4260 |
| ccacgctttg cctgaccctg cttgctcaac tctacgtctt tctttcgttt tctgttctgc | 4320 |
| gccgttacag atccaagctg tgaccggcgc ctacgcggcc gcaccatgaa ctacgagggt | 4380 |
| cacctgaagg gccaccgcgg atgggtcacc tccctggcct gcccgcagca ggcggggtcg | 4440 |
| tacatcaagg tggtgtcgac gtcgcgcgat ggcacggcca tctcgtggaa agccaacccc | 4500 |
| gaccgccaca gcgtggacag cgactacggt ctgccgagcc accgcctcga ggccacaccg | 4560 |
| gcttcgtgtc gtgtgtgtcg ctggcccacg ccaccgacta cgcgctgacc gcgtcctggg | 4620 |
| accgctccat ccgcatgtgg gacctgcgca atggccagtg ccagcgcaag ttcctgaagc | 4680 |
| acaccaagga cgtgctcgcc gtcgccttct cgccggacga ccgcctgatc gtgtccgccg | 4740 |
| gccgcgacaa cgtgatccgc gtgtggaacg tggcgggcga gtgcatgcac gagttcctgc | 4800 |
| gcgacggcca cgaggactgg gtgagcagca tctgtttctc gccgtcgctg gagcatccga | 4860 |
| tcgtggtgtc cggcagctgg gacaacacca tcaaggtatg gaacgtgaac gggggcaagt | 4920 |
| gtgagcgcac gctcaagggc cacagcaact acgtgtccac ggtgacggtg tcgccagacg | 4980 |
| ggtcgctgtg cgcgtccggc ggcaaggacg gcgcggcgct gctgtgggac ctgagcaccg | 5040 |
| gcgagcagct gttcaagatc aacgtggagt cgcccatcaa ccagatcgcc ttctcgccca | 5100 |
| accccttctg gatgtgcgtc gcgacggaga ggtctctgtc cgtgtacgac ctggagagca | 5160 |
| aggctgtgat tgcggagctg acgccggacg gcgcgaagcc gtccgagtgc atctccattg | 5220 |
| cctggtccgc cgacggcaac actctgtact ccggtcacaa ggacaacctg atccgcgtgt | 5280 |
| ggtccatctc cgacgccgag taagaattcc cagacatgat aagatacatt gatgagtttg | 5340 |
| gacaaaccac aactagaatg cagtgaaaaa aatgctttat ttgtgaaatt tgtgatgcta | 5400 |
| ttgctttatt tgtaaccatt ataagctgca ataaacaagt taacaacaac aattgcattc | 5460 |
| attttatgtt tcaggttcag ggggaggtgt gggaggtttt ttaaagcaag taaaacctct | 5520 |
| acaaatgtgg tatgtccatg acgttcctga ccttcgtgca tcgatgcagg gggtgactgt | 5580 |
| gaacgttcga gatgatcgtc gaacgttcga gatgatgaat cccggtgcta aaacttcatt | 5640 |
| tttaatttaa aaggatctag gtgaagatcc tttttgataa tctcatgacc aaaatccctt | 5700 |
| aacgtgagtt ttcgttccac tgagcgtcag accccgtaga aaagatcaaa ggatcttctt | 5760 |
| gagatccttt ttttctgcgc gtaatctcct gcttgcaaac aaaaaaacca ccgctaccag | 5820 |
| cggtcgtttg tttgccggat caagagctac caactctttt tccgaaggta actggcttca | 5880 |
| gcagagcgca gataccaaat actctccttc tagtgtagcc gtagttaggc caccacttca | 5940 |
| agaactctgt agcaccgcct acatacctcg ctctgctaat cctgttacca gtcgctgctg | 6000 |
| ccagtggcga taagtcgtgt cttaccgggt tggactcaag acgatagtta ccggataagg | 6060 |
| cgcagcggtc gggctgaacg gggggttcgt gcacacagcc cagcttggag cgaacgacct | 6120 |
| acaccgaact gagataccta cagcgtgagc tatgagaaag cgccacgctt cccgaaggga | 6180 |
| gaaaggcgga caggtatccg gtaagcggca gcgtcggaac aggagagcgc acgagggagc | 6240 |
| ttccaggggg aaacgcctgg tatcttatag tcctgtcggg tttcgccacc tctgacttga | 6300 |
| gcgtcgattt ttgtgatgct cgtcaggggg gcggagccta tggaaaaacg ccagcaacgc | 6360 |
| ggccttttta ccgttcctgg gcttttgctg gccttttgct cacatgttct t | 6411 |

```
The amino acid sequence SEQ ID No. 4:
Met Ala Lys Thr Ile Ala Tyr Asp Glu Glu Ala Arg Arg Gly Leu Glu
1 5 10 15
Arg Gly Leu Asn Ser Leu Ala Asp Ala Val Lys Val Thr Leu Gly Pro
                        20 25 30
Lys Gly Arg Asn Val Val Leu Glu Lys Lys Trp Gly Ala Pro Thr Ile
                35 40 45
Thr Asn Asp Gly Val Ser Ile Ala Lys Glu Ile Glu Leu Glu Asp Pro
        50 55 60
Tyr Glu Lys Ile Gly Ala Glu Leu Val Lys Glu Val Ala Lys Lys Thr
65 70 75 80
Asp Asp Val Ala Gly Asp Gly Thr Thr Thr Ala Thr Val Leu Ala Gln
                                85 90 95
Ala Leu Val Lys Glu Gly Leu Arg Asn Val Ala Ala Gly Ala Asn Pro
                        100 105 110
Leu Gly Leu Lys Arg Gly Ile Glu Lys Ala Val Asp Lys Val Thr Glu
                115 120 125
Thr Leu Leu Lys Asp Ala Lys Glu Val Glu Thr Lys Glu Gln Ile Ala
        130 135 140
Ala Thr Ala Ala Ile Ser Ala Gly Asp Gln Ser Ile Gly Asp Leu Ile
145 150 155 160
Ala Glu Ala Met Asp Lys Val Gly Asn Glu Gly Val Ile Thr Val Glu
                                165 170 175
Glu Ser Asn Thr Phe Gly Leu Gln Leu Glu Leu Thr Glu Gly Met Arg
                        180 185 190
Phe Asp Lys Gly Tyr Ile Ser Gly Tyr Phe Val Thr Asp Ala Glu Arg
                195 200 205
Gln Glu Ala Val Leu Glu Glu Pro Tyr Ile Leu Leu Val Ser Ser Lys
        210 215 220
Val Ser Thr Val Lys Asp Leu Leu Pro Leu Leu Glu Lys Val Ile Gln
225 230 235 240
Ala Gly Lys Ser Leu Leu Ile Ile Ala Glu Asp Val Glu Gly Glu Ala
                                245 250 255
Leu Ser Thr Leu Val Val Asn Lys Ile Arg Gly Thr Phe Lys Ser Val
                        260 265 270
Ala Val Lys Ala Pro Gly Phe Gly Asp Arg Arg Lys Ala Met Leu Gln
                275 280 285
Asp Met Ala Ile Leu Thr Gly Ala Gln Val Ile Ser Glu Glu Val Gly
        290 295 300
Leu Thr Leu Glu Asn Thr Asp Leu Ser Leu Leu Gly Lys Ala Arg Lys
305 310 315 320
Val Val Met Thr Lys Asp Glu Thr Thr Ile Val Glu Gly Ala Gly Asp
                                325 330 335
Thr Asp Ala Ile Ala Gly Arg Val Ala Gln Ile Arg Thr Glu Ile Glu
                        340 345 350
Asn Ser Asp Ser Asp Tyr Asp Arg Glu Lys Leu Gln Glu Arg Leu Ala
                355 360 365
Lys Leu Ala Gly Gly Val Ala Val Ile Lys Ala Gly Ala Ala Thr Glu
        370 375 380
Val Glu Leu Lys Glu Arg Lys His Arg Ile Glu Asp Ala Val Arg Asn
385 390 395 400
Ala Lys Ala Ala Val Glu Glu Gly Ile Val Ala Gly Gly Gly Val Thr
                                405 410 415
Leu Leu Gln Ala Ala Pro Ala Leu Asp Lys Leu Lys Leu Thr Gly Asp
                        420 425 430
Glu Ala Thr Gly Ala Asn Ile Val Lys Val Ala Leu Glu Ala Pro Leu
                435 440 445
Lys Gln Ile Ala Phe Asn Ser Gly Met Glu Pro Gly Val Val Ala Glu
        450 455 460
Lys Val Arg Asn Leu Ser Val Gly His Gly Leu Asn Ala Ala Thr Gly
465 470 475 480
Glu Tyr Glu Asp Leu Leu Lys Ala Gly Val Ala Asp Pro Val Lys Val
                                  485 490 495
Thr Arg Ser Ala Leu Gln Asn Ala Ala Ser Ile Ala Gly Leu Phe Leu
                        500 505 510
Thr Thr Glu Ala Val Val Ala Asp Lys Pro Glu Lys Thr Ala Ala Pro
                515 520 525
Ala Ser Asp Pro Thr Gly Gly Met Val Thr Ser Gly His Asp Ala Gly
        530 535 540
Ser Tyr Val Val
545
```

### EXAMPLES

### Example 1: Construction of Plasmid pVAX_Hsp65_Lack

Construction of pVAX_hsp65 plasmids containing hsp65 gene was constructed from the pVAX vector (Invitrogen, Carlsbad, CA, USA). This plasmid was digested with BamHI and NotI (Gibco BRL, Gaithersburg, MD, USA) and then the M. leprae hsp65 gene according to SEQ ID No. 4, comprising the amino acids 48-483 of SEQ ID No. 1, and the CMV intron A were inserted. DH5α E. coli transformed with pVAX or pVAXhsp65 were cultured in LB liquid medium (Gibco BRL, Gaithersburg, MD, USA) containing kanamycin (100 μg/ml). The plasmids were purified using the Concert High Purity Maxiprep System (Gibco BRL, Gaithersburg, MD, USA). Plasmid concentrations were determined by spectrophotometry at λ=260 and 280 nm by using the Gene Quant II apparatus (Pharmacia Biotech, Buckinghamshire, UK).

For the construction of the Leishmania DNA vaccine, based on plasmid pVAX_hsp65, a promoter named EF1a/HTLV was inserted where the Lack gene could be inserted having as a restriction site the sequences for the NotI and EcoRI enzymes (GCGGCCGC—NotI enzyme site/GAATTC—EcoRI). EF1a-HTLV promoter is a composite promoter comprising the human Elongation Factor-1α (EF-1α) core promoter and the R segment and part of the U5 sequence (R-U5′) of the Human T-Cell Leukemia Virus (HTLV) Type 1 Long Terminal Repeat. Thus, the final construction of the DNA plasmid was based on the insertion of the hsp65 and the Lack gene into different promoters: CMV promoter for hsp65 and EF1a/HTLV for Lack.

The great advantage of these plasmid systems is that it allows to maintain the immunomodulatory activities of hsp65 and count on the insertion of the LACK immunogenic gene for use in the specific protection of dogs against infection caused by Leishmania.

To increase the immunogenicity of the multipurpose plasmid system, 4 CpGs Motifs were also added after the SV40 polyadenylation site of the LACK gene expression cassette.

Constructs of DNA plasmids containing only Lack gene, without the hsp65 gene, were also produced and were used as controls in immunogenicity and vaccine efficacy studies. Thus, the following plasmid DNAs were constructed:
(i) pVAX_hsp65 (control)
(ii) pVAX_lack (control)
(iii) pVAX_hsp65_lack
(iv) pVAX_A2 (reference control)
(v) pVAX_hsp65_A2 (reference control).

To obtain recombinant proteins (LACK) and specific antibodies (anti-LACK), plasmids pET28a_lack and pET28a_A2 were manufactured by standard methods. These plasmids were used for transformation of E. coli lineage BL21 strains for expression of the Lack protein. Proteins were used in the production of anti-Lack polyclonal antibodies, and in the immunogenicity, evaluation experiments of DNA vaccines in mice and dogs. The production of anti-Lack polyclonal antibodies was made in mice by immunization with each of the proteins administered together with Freud's Incomplete Adjuvant. The plasmids pVAX_hsp65, pVAX_hsp65_lack, pVAX_lack, were transformed into E. coli TOP10 lineage for purification and characterization of the cloned genes in each plasmid through polyacrylamide gel analysis after digestion with restriction enzymes.

Following laboratory scale production, transfection of HEK293 cells with each of the plasmids and demonstration of the endogenous hsp65 and lack proteins in the respective vaccine constructs were performed. Expression of proteins was characterized by Western Blot techniques using anti-hsp65 and anti-LACK polyclonal antibodies.

mRNA was detected by RT-PCR using specific primers. The pVAX_hsp65, pVAX_hsp65_lack and pVAX_lack, vaccines were produced on a laboratory scale in the amount around of 10 mg using standard methodology. After bench scale production, DNA plasmids were characterized as to: identity by restriction enzyme digestion; appearance; concentration; purity (A260/280 between 1.7-2.0); homogeneity (>90% in supercoiled form); Genomic DNA (<5%); and endotoxin (Guidance for industry-FDA-2007<40 EU/mg). After the characterization of each of the vaccines they were used for immunogenicity studies and efficacy in mice. After laboratory scale production and quality control tests the vaccine constructs pVAX_hsp65, pVAX_hsp65_lack and pVAX_lack were ready to be tested for immunogenicity and efficacy in the experimental model of leishmaniasis in mice.

Cloning of the lack gene on plasmid pVAX_hsp65 was performed by the GeneScript company generating the plasmid pVAX_hsp65_lack. Together with the lack gene the promoter region (Double EF1α/HTL promoter), the polyadenylation signal (SV40) and the CPGs sequences were also cloned as described above using standard techniques.

The resulting plasmid pVAX_hsp65_lack is according to SEQ ID NO. 3 and has the schematic representation of FIG. 1.

Plasmids (iv) and (v) containing the A2 antigen were constructed according to the same procedures as described above for the LACK containing antigens. DNAhsp65 was used as the vaccine platform (prototype), which already has established immunomodulatory activities, and added to that plasmid the A2 encoding gene of Leishmania to result in construct pVAX_hsp65_A2 (v). The plasmid pVAX_A2 (iv) was constructed by removing the hsp65 gene from plasmid pVAX_hsp65_A2 by the company GeneScript.

### Example 2: Characterization of Plasmid pVAX_Hsp65_Lack

The plasmid pVAX_hsp65_lack constructed by the GeneScript Company was transformed into E. coli lineage TOP10 for propagation and storage. The plasmid was characterized by restriction analysis with the NotI and EcoRI enzymes, which were added at the ends of the lack gene during the construct, and with the BamHI and ApaI enzymes used in the cloning of the hsp65 gene. In the digestion, 1 μg of the plasmid (for each digestion), 1 μL of each enzyme was used in a final 20 μL reaction incubated at 37° C. for a time of 30 minutes to 1 hour. The digestion evaluation was performed on 1% agarose gel and with addition of GelRed in the samples.

The plasmid pVAX_hsp65_lack was also used for transfection of HEK293 cells and evaluation of the lack and hsp65 protein expression by Western blot and identification of mRNA for lack and hsp65 by RT-PCR using specific primers. Transfection of HEK cells with the plasmid pVAX_hsp65_lack was performed to evaluate the ability of this new plasmid construct to generate transcripts (mRNA) and protein. After 24 hours of culture of HEK cells in the presence of DNA plasmids, the medium was removed, cells washed with PBS (Phosphate Buffered Saline) and Trizol (Invitrogen) added. Cells were homogenized, collected and frozen at −70° C. RNA extraction was performed according to manufacturer's instructions. After extraction and quantification, 1 μg of RNA was treated with DNAse I and submitted to the reaction for cDNA production. RNA from all transfections (with pVAX, pVAX_hsp65 and pVAX_hsp65_lack) was treated in the same manner.

Detection of the transcript encoded by the plasmid pVAX_hsp65_lack was performed by the polymerase chain reaction (PCR) using specific oligonucleotides. PCR-specific primers as a positive control reaction were used for GAPDH (glyceraldehyde-3-phosphate dehydrogenase) primers. After 24 hours of culture of the HEK cells in the presence of the DNA plasmids, the medium was removed, cells washed with PBS and collected with PBS or CelLyticM (Sigma) for evaluation by Western Blot.

The results showed that the plasmid pVAX_hsp65_lack was constructed correctly since the genes lack and hsp65 were characterized in plasmids pVAX_hsp65_lack by restriction enzymes specific for the cloning sites of the respective genes. Lack and hsp65 mRNA expression were characterized by PCR in HEK cells transfected with the pVAX_hsp65_lack. Hsp65 and lack proteins were also characterized by Western blot analysis in the supernatant of HEK cells transfected with pVAX_hsp65_lack plasmid.

### Example 3: Immunogenicity and Efficacy of pVAX_Hsp65_Lack in Murine Model

In a murine model distinct plasmid constructions and combinations resuspended in saline were tested. For that, it was designed an experiment to evaluate immunogenicity and efficacy post experimental infection in mice (Mus musculus—Balb/c). A total of 128 mice were randomly divided into eight experimental groups, as follow:
Group A—pVAX_hsp65 (control)
Group B—pVAX_A2 (reference control)
Group C—Leish-Tec® (control)
Group D—pVAX_hsp65_lack
Group E—pVAX_hsp65_A2(reference control)
Group F—pVAX_hsp65_lack+pVAX_hsp65_A2
Group G—pVAX_lack (control)
Group H—saline (negative control)

The experimental groups were then separated in two (immunogenicity and efficacy), with eight animals each. Animals were vaccinated with three doses, applied subcutaneously with 15 days between each dose. Then, 15 days post the 3^{rd} vaccination dose, the animals were experimentally infected with 10⁸ parasites of L. infantum chagasi, cepa NCL/IOCL 3241 through intraperitoneal route (FIG. 2).

For immunogenicity evaluation 15 days post the 3^{rd} vaccine dose, blood samples were collected for antigenic specific (Hsp65, Lack and A2) antibody IgG1 and IgG2 quantification through ELISA test. Additionally, animals' spleen samples (8 animals per group) were collected for IFN-g quantification in commercial kits, following manufacturer instructions.

The vaccines formulations efficacy was evaluated by parasite burden in liver and spleen using qPCR and limiting dilution assay, 30 days post experimental infection.

### Results:

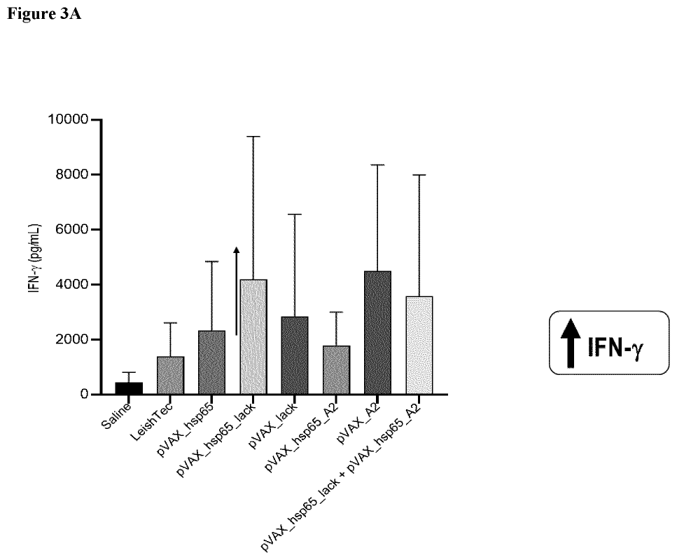

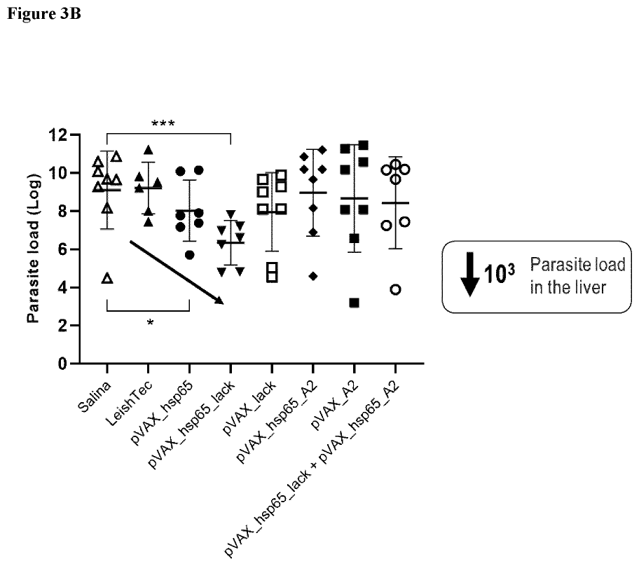

The immunogenicity results indicated an antibody response in vaccinated groups. An increase of the IFN-γ response in the group vaccinated with pVAX_hsp65_lack was observed (FIG. 3A), indicating a Th1 modulatory response.

In particular, the antibody response was about twice as high for the pVAX_hsp65_lack vaccine as for the commercial vaccine Leish-Tec. In addition, the IFN-γ cellular immune response was significantly higher compared to DNA vaccines using the same pVAX plasmid backbone but including only one of hsp65 and Lack gene. Moreover, Lack as antigen achieved a high IFN-γ response when combined with the immunomodulator hsp65. Instead, the vaccine including the A2 antigen, although achieving a similar antibody response as the vaccine pVAX_hsp65_lack, did not benefit from its combination with the hsp65 immunomodulator, since the IFN-γ response was lower for the hsp65_A2 combination construct compared with the A2 construct not containing the immunomodulator. Interestingly, the combination of both combination constructs pVAX_hsp65_lack and pVAX_hsp65_A2 did not result in an increased antibody response compared to the single constructs. Additionally, in efficacy evaluation, the parasite load in the liver of the vaccinated group pVAX_hsp65_lack after 30 days of challenge was 10³ units lower than non-vaccinated (saline) group (FIG. 3B).

In particular, the reduction in parasite load was higher for the pVAX_hsp65_lack vaccine as for the commercial vaccine Leish-Tec, which did not achieve any significant reduction in parasite load. In addition, the parasite load was lower compared to DNA vaccines using the same pVAX plasmid backbone but including only one of hsp65 and Lack gene. In addition, no significant reduction in parasite load could be observed for any of the comparative vaccines using the A2 antigen. Therefore, although the pVAX_A2 vaccine achieved a similar antibody response than the vaccine pVAX_hsp65_lack, this antibody response did not result in any significant reduction of parasite load. The combination of both combination constructs pVAX_hsp65_lack and pVAX_hsp65_A2 also did not result in any significant reduction of parasite load compared to the single constructs, mirroring the measured antibody response.

In conclusion, the murine model experiment indicated that the immune response and protection observed in the group vaccinated with pVAX_hsp65_lack antigen were superior to the other experimental groups. In particular, it was surprisingly observed that antibody and cellular immune response and parasite load were superior in the group vaccinated with pVAX_hsp65_lack compared with the group vaccinated with pVAX_hsp65_A2, indicating that superior results can be achieved with the combination of the hsp65 immunomodulator and LACK antigen compared with the combination of hsp65 and the A2 antigen. This result is particularly surprising, since it was recently shown in the art that an immune response induced by the LACK antigen does not provide protection against experimental Leishmania infection and is weaker than with the A2 antigen (Coelho et al., Infection and Immunity, 71(7), 2003).

In addition, it could be shown that the DNA vaccine of the invention achieves an increases immune response (cellular and humoral) and reduced parasite load compared to subjects vaccinated with the prior art vaccine Leish-Tec®, a commercial vaccine based on the A2 antigen of Leishmania, showing remarkable improvement compared to prior art vaccines.

### Example 4: Immunogenicity and Efficacy of pVAX_Hsp65_Lack in Canine Model

The aim of this study is to assess the efficacy and safety of experimental vaccines against Leishmania infantum in dogs.

Forty-eight healthy conventional puppies (males and females), previously castrated, aged between 6 and 12 months old, negative for L. infantum in serology and bone marrow PCR were included in the study. The study was masked and randomized, consisting of four groups containing 10 to 11 animals each. Each animal represents an experimental unit. The animals were randomly allocated to experimental groups according to the age, body weight, and sex. Two experimental vaccines against L. infantum were tested and their results were compared to negative and positive control groups (a competitor vaccine available on the Brazilian market).

All groups were vaccinated in a three doses protocol administered 21 days apart by subcutaneous route (Table 1).

TABLE 1
Treatment groups and vaccine schedule.

| | | | Route of | Number |
|---|---|---|---|---|
| | | Vaccination | Adminis- | of |
| Group | Vaccine | regimen | tration | Animals |
| 1 | pVAX_Lack_hsp65 + | 1^{st} dose: | Subcu- | 11 |
| | saponin | D 0 | taneous | |
| 2 | pVAX_Lack_hsp65 + | 2^{nd} dose: | (1 mL) | 11 |
| | saline | D 21 | | |
| 3 | Leish-Tec − Ceva | 3^{rd} dose: | | 11 |
| | Saúde ® Animal | D 42 | | |
| 4 | Negative control (saline) | | | 10 |

All animals were challenged with L. infantum inoculum fourteen weeks after the third dose of vaccine by intravenous route. From this moment, the animals were monitored for the occurrence of clinical changes related to canine leishmaniasis, considering laboratory markers and clinical signs.

For immunogenicity evaluation of the humoral and cellular immune response to the vaccines, blood samples were collected on Day 21 (before the second dose), Day 42 (before the third dose), Day 56, Day 141 (just prior to the challenge), Day 184, Day 219, Day 254, Day 331, Day 388 and Day 443.

Aspiration punctures from bone marrow and lymph nodes (popliteal or prescapular) were performed from each animal for searching of L. infantum DNA in order to evidence the absence of infection by the parasite in the animals before the challenge or to quantify the parasite load after the challenge. Bone marrow samples were collected according to the methodology of Paparcone et al. (2013). Bone marrow and popliteal lymph nodes samples were obtained on Day 56 and Day 141 to evidence the absence of L. infantum genetic material just before the challenge. After this, samples from the same tissue were collected on Day 184, Day 254, Day 331, Day 388 and Day 443 in order to quantify the parasite load by real time qPCR.

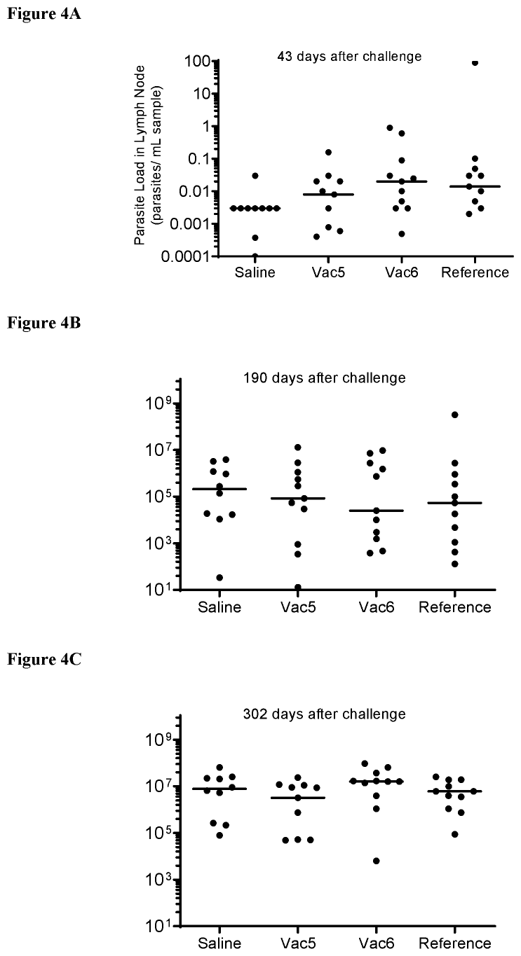

FIGS. 4A, 4B and 4C show parasite load by qPCR in the lymph node (individual data and median) for days 43, 190 and 302 after challenge. The saline group received no vaccination, groups Vac5 and Vac6 received pDNA vaccine according to the invention (pVAX_hsp65_lack) and Reference group received commercially available Leish-Tec. Vac5 is a vaccine with pDNA and saponin, Vac6 is a vaccine with pDNA and saline. To quantify the parasite load of L. infantum, qPCR was used employing primers directed at the circular DNA of the parasite kinetoplast (kDNA) (direction: 5′-GTGGGGGGAGGGGCGTTCT-3′ (SEQ ID NO: 5) and reverse: 5′-ATTTTACACCAACCCCCAGTT-3′ (SEQ ID NO: 6)). The qPCR reaction was standardized with 10 ng of purified genomic DNA using SYBR@Green probe.

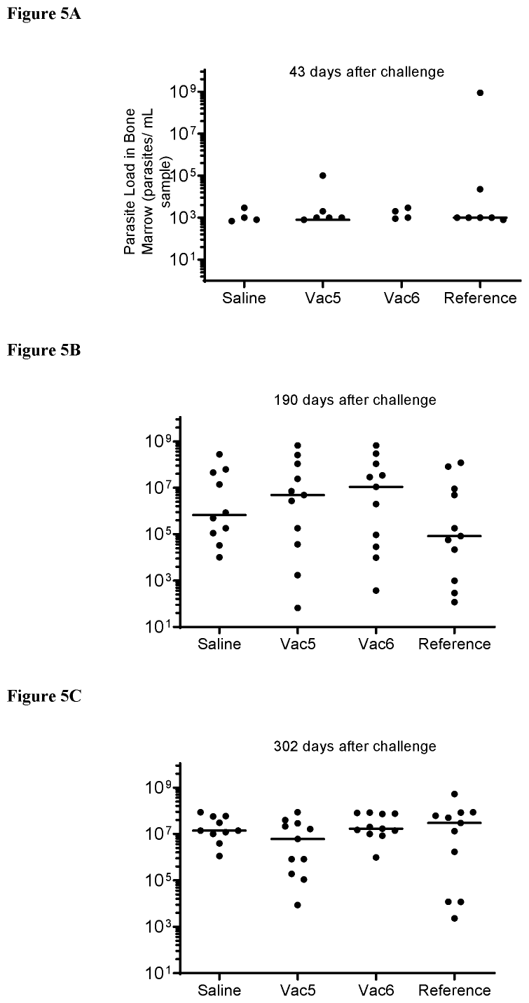

FIGS. 5A, 5B and 5C show parasite load by qPCR in the bone marrow (individual data and median) for days 43, 190 and 302 after challenge for the same groups of animals as described for FIG. 4.

Clinical examination was done on days 247 and 302 post Leishmania challenge. Some examples of clinical signs included ear dermatitis, onychogryphosis, lymphadenomegaly, muscle atrophy, ear dermatitis, flaking skin, alopecia, depigmentation, skin ulcers, hyperkeratosis, blepharitis. It is evident that the challenge model used can cause clinical changes in the animals, a situation that highlights the potential protection of the vaccines used in the study. Animals with such alterations were observed in all experimental groups, with a difference in the frequency of more or less clinically affected animals in each of the groups. examination and alterations found in the laboratory markers that are associated with canine leishmaniasis. This proved to be the primary endpoint to highlight a potential protective effect of the tested vaccine formulations.

TABLE 2
Frequency of animals per group

| Classification | n | Saline | Reference | Vac5 | Vac6 |
|---|---|---|---|---|---|
| Symptomatic | Presence of ≥2 significant | 5/10 (50%) | 7/11 (63%) | 4/11 (36%) | 3/11 (27%) |
| disease | clinical signs, with or | | | | |
| | without lab changes | | | | |
| | (hemogram and biochemistry) | | | | |
| | Presence of 1 significant | 2/10 (20%) | 1/11 (10%) | 1/11 (10%) | 3/11 (27%) |
| | clinical signs, with ≥2 lab | | | | |
| | changes (hemogram and | | | | |
| | biochemistry) | | | | |
| Asymptomatic | Absence of clinical signs | 3/10 (30%) | 3/11 (27%) | 6/11 (54%) | 5/11 (45%) |
| disease | with minor lab changes or | | | | |
| | absence on lab markers | | | | |

As can be seen in Table 2, the groups received the vaccine according to the invention (Vac5, Vac6) show the lowest number of animals with a symptomatic disease and the highest number of animals with an asymptomatic disease.

It can be concluded that while the parasite load in lymph nodes and bone marrow is similar across all dog groups until 302 days after the challenge, the experimental vaccines based on plasmid pVAX_Lack_Hsp65 reduced the severity of the disease, when comparing the clinical signs and laboratory changes obtained for the saline group and the group vaccinated with a reference vaccine.

1. An isolated polynucleotide comprising:
(i) a first expression cassette comprising a nucleic acid encoding heat shock protein (hsp) 65, or a functional fragment thereof, and
(ii) a second expression cassette comprising a nucleic acid encoding LACK (Leishmania homologue of receptors for activated C kinase), or an immunogenic fragment thereof.

2. The polynucleotide of claim 1, wherein hsp 65 comprises the amino acids 48 to 583 of the amino acid sequence according to SEQ ID NO:1 or comprises an amino acid sequence having at least 90% sequence identity thereof.

3. The polynucleotide of claim 1 or 2, wherein LACK comprises the amino acid sequence according to SEQ ID NO:2 or comprises an amino acid sequence having at least 90% sequence identity thereof.

4. The polynucleotide of any one of the preceding claims, wherein the nucleic acids encoding hsp65 and LACK are under control of different promoters.

5. The polynucleotide of any one of the preceding claims, wherein the nucleic acid encoding hsp65 is under control of a cytomegalovirus (CMV) promoter.

6. The polynucleotide of any one of the preceding claims, wherein the nucleic acid encoding LACK is under the control of an EF1α-HTLV promoter.

7. The polynucleotide of any one of the preceding claims, wherein the second expression cassette further comprises one or more CpG motifs.

8. A DNA plasmid comprising the polynucleotide of any one of claims 1-7.

9. Use of the polynucleotide of any one of claims 1 to 7 or of the DNA plasmid of claim 8 for the recombinant expression of hsp65 and LACK or immunogenic fragments thereof.

10. A DNA vaccine comprising the polynucleotide of any one of claims 1 to 7 or comprising the DNA plasmid of claim 8.

11. The DNA vaccine of claim 10, further comprising a pharmaceutically acceptable carrier.

12. The DNA vaccine of claim 10 or 11, which is a non-adjuvated vaccine.

13. The DNA vaccine of claim 10 or 11, which comprises an adjuvant, preferably wherein the adjuvant is saponin.

14. The DNA vaccine of any one of claims 10 to 13 for use in the protection of a subject against an infection with leishmaniasis, preferably wherein the subject is dog.

15. The DNA vaccine for use of claim 14, wherein the infection is with canine leishmaniasis.


SEQUENCE LISTING (6 sequences)

SEQ ID NO: 1 - protein, 588 aa, Mycobacterium leprae
  source 1..588: note = Heat shock protein Hsp65
MPGRDGETQPASCGRPSRALHPASVSNGGCRHPVTLASFLIRRNHFAMAKTIAYDEEARRGLERGLNSLADAVKVTLGPKGRNVVLEKKWGAPTITNDGVSIAKEIELEDPYEKIGAELVKEVAKKTDDVAGDGTTTATVLAQALVKEGLRNVAAGANPLGLKRGIEKAVDKVTETLLKDAKEVETKEQIAATAAISAGDQSIGDLIAEAMDKVGNEGVITVEESNTFGLQLELTEGMRFDKGYISGYFVTDAERQEAVLEEPYILLVSSKVSTVKDLLPLLEKVIQAGKSLLIIAEDVEGEALSTLVVNKIRGTFKSVAVKAPGFGDRRKAMLQDMAILTGAQVISEEVGLTLENTDLSLLGKARKVVMTKDETTIVEGAGDTDAIAGRVAQIRTEIENSDSDYDREKLQERLAKLAGGVAVIKAGAATEVELKERKHRIEDAVRNAKAAVEEGIVAGGGVTLLQAAPALDKLKLTGDEATGANIVKVALEAPLKQIAFNSGMEPGVVAEKVRNLSVGHGLNAATGEYEDLLKAGVADPVKVTRSALQNAASIAGLFLTTEAVVADKPEKTAAPASDPTGGMGGMDF

SEQ ID NO: 2 - protein, 312 aa, Leishmania infantum
  source 1..312: note = Leishmania activated protein kinase C receptor - LACK
MNYEGHLKGHRGWVTSLACPQQAGSYIKVVSTSRDGTAISWKANPDRHSVDSDYGLPSHRLEGHTGFVSCVSLAHATDYALTASWDRSIRMWDLRNGQCQRKFLKHTKDVLAVAFSPDDRLIVSAGRDNVIRVWNVAGECMHEFLRDGHEDWVSSICFSPSLEHPIVVSGSWDNTIKVWNVNGGKCERTLKGHSNYVSTVTVSPDGSLCASGGKDGAALLWDLSTGEQLFKINVESPINQIAFSPNRFWMCVATERSLSVYDLESKAVIAELTPDGAKPSECISIAWSADGNTLYSGHKDNLIRVWSISDAE

SEQ ID NO: 3 - DNA, 6411 bp, synthetic construct
  source 1..6411: mol_type = other DNA; note = Hsp65_Lack Expression Construct
gactcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctactagagaacccactgcttactggcttatcgaaattaatacgactcactatagggagacccaagctggctagcgtttaaacttaagcttggtaccgagctcggatccgatacttcgcaatggccaagacaattgcgtacgacgaagaggcccgtcgcggcctcgagcggggcttgaacagcctcgccgacgcggtaaaggtgacgttgggtccgaaggggcgcaacgtcgttctagagaagaagtggggtgctcccacgatcaccaacgatggcgtgtccatcgccaaggagatcgagctggaggacccgtacgagaagattggcgctgagttggtcaaggaagtcgccaagaagacagatgacgtcgccggtgatggcaccacgacggccaccgtgctggcccaggcattggtcaaagagggcctacgcaacgtcgcggccggcgccaacccgctaggtctcaagcgtggcatcgagaaagctgtcgataaggtaactgagactctgctcaaggacgctaaggaggtcgaaaccaaggaacaaattgctgccactgcagcgatttcggcgggtgaccagtcgatcggtgatctgatcgccgaggcgatggacaaggttggcaacgagggtgttatcaccgtcgaggaatccaacaccttcggtctgcagctcgagctcaccgagggaatgcggttcgacaagggctacatttcgggctacttcgtcaccgacgccgagcgtcaggaagctgtcctagaggagccctacatccttctggtcagctccaaagtgtctaccgtcaaggacctgctgccgctgctagagaaggtcatccaggccggcaagtcgctgctgatcattgctgaggatgtcgagggtgaggcgttgtctaccctggtcgtcaacaagatccgtggcactttcaagtcggtggcggtcaaagctcctggctttggtgaccgccgcaaggcaatgttgcaagacatggccattctcaccggagcccaggtcatcagcgaggaggtcggtctcacattggagaacaccgatctgtcattgctgggcaaggcccgcaaggtggttatgaccaaggacgaaaccaccatcgtcgagggtgccggtgacaccgacgccatcgccgggcgagtggctcagatccgtaccgagatcgagaacagtgactctgactatgaccgcgagaaactgcaggaacgcctggctaagttggccggtggtgttgcggtgatcaaggccggtgctgccactgaggtggagctcaaggagcgcaagcaccgcatcgaggacgcagtccgcaacgccaaggccgcggtggaggaggggatcgtcgccggcggcggtgtgactctgctacaggctgctccggctctggacaagctgaagctgaccggtgacgaggcgaccggtgccaatattgtcaaggtggcgttggaagctccgctcaagcagatcgccttcaattccgggatggagcccggcgtggtggccgaaaaggtgcgtaacctttcagtgggtcacggcctgaacgccgccaccggtgagtacgaggacctgctcaaggccggcgttgccgacccggtgaaggttacacgttctgcgctgcagaacgcagcgtccatcgccggcctgttccttactacggaggccgtcgtcgccgacaagccggagaagacggcagctccggcgagcgacccgaccggtggcatggtgacgtccggtcatgatgcaggtagctacgtggtctgagggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctactgggcggttttatggacagcaagcgaaccggaattgccagctggggcgccctctggtaaggttgggaagccctgcaaagtaaactggatggctttctcgccgccaaggatctgatggcgcaggggatcaagctctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaagacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgagcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgaattattaacgcttacaatttcctgatgcggtattttctccttacgcatctgtgcggtatttcacaccgcatacaggtggcacttttcggggaaatgtgcgcggaacccctatttgtttatttttctaaatacattcaaatatgtatccgctcatgagacaataaccctgataaatgcttcaataatagcaccccgggatctgcgatcgctccggtgcccgtcagtgggcagagcgcacatcgcccacagtccccgagaagttggggggaggggtcggcaattgaacgggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacagctgaagcttcgaggggctcgcatctctccttcacgcgcccgccgccctacctgaggccgccatccacgccggttgagtcgcgttctgccgcctcccgcctgtggtgcctcctgaactgcgtccgccgtctaggtaagtttaaagctcaggtcgagaccgggcctttgtccggcgctcccttggagcctacctagactcagccggctctccacgctttgcctgaccctgcttgctcaactctacgtctttgtttcgttttctgttctgcgccgttacagatccaagctgtgaccggcgcctacgcggccgcaccatgaactacgagggtcacctgaagggccaccgcggatgggtcacctccctggcctgcccgcagcaggcggggtcgtacatcaaggtggtgtcgacgtcgcgcgatggcacggccatctcgtggaaagccaaccccgaccgccacagcgtggacagcgactacggtctgccgagccaccgcctcgaggccacaccggcttcgtgtcgtgtgtgtcgctggcccacgccaccgactacgcgctgaccgcgtcctgggaccgctccatccgcatgtgggacctgcgcaatggccagtgccagcgcaagttcctgaagcacaccaaggacgtgctcgccgtcgccttctcgccggacgaccgcctgatcgtgtccgcgggccgcgacaacgtgatccgcgtgtggaacgtggcgggcgagtgcatgcacgagttcctgcgcgacggccacgaggactgggtgagcagcatctgtttctcgccgtcgctggagcatccgatcgtggtgtccggcagctgggacaacaccatcaaggtatggaacgtgaacgggggcaagtgtgagcgcacgctcaagggccacagcaactacgtgtccacggtgacggtgtcgccagacgggtcgctgtgcgcgtccggcggcaaggacggcgcggcgctgctgtgggacctgagcaccggcgagcagctgttcaagatcaacgtggagtcgcccatcaaccagatcgccttctcgcccaaccgcttctggatgtgcgtcgcgacggagaggtctctgtccgtgtacgacctggagagcaaggctgtgattgcggagctgacgccggacggcgcgaagccgtccgagtgcatctccattgcctggtccgccgacggcaacactctgtactccggtcacaaggacaacctgatccgcgtgtggtccatctccgacgccgagtaagaattcccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtatgtccatgacgttcctgacgttggtgcatcgatgcagggggtgactgtgaacgttcgagatgatcgtcgaacgttcgagatgatgaatcccggtgctaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatcttatagtcctgtcgggtttcgccacctctgacttgagcgtcgatttttgtgatgctcgtcaggggggcggagcctatggaaaaacgccagcaacgcggcctttttacggttcctgggcttttgctggccttttgctcacatgttctt

SEQ ID NO: 4 - protein, 548 aa, synthetic construct
  source 1..548: note = Hsp65_fragment
MAKTIAYDEEARRGLERGLNSLADAVKVTLGPKGRNVVLEKKWGAPTITNDGVSIAKEIELEDPYEKIGAELVKEVAKKTDDVAGDGTTTATVLAQALVKEGLRNVAAGANPLGLKRGIEKAVDKVTETLLKDAKEVETKEQIAATAAISAGDQSIGDLIAEAMDKVGNEGVITVEESNTFGLQLELTEGMRFDKGYISGYFVTDAERQEAVLEEPYILLVSSKVSTVKDLLPLLEKVIQAGKSLLIIAEDVEGEALSTLVVNKIRGTFKSVAVKAPGFGDRRKAMLQDMAILTGAQVISEEVGLTLENTDLSLLGKARKVVMTKDETTIVEGAGDTDAIAGRVAQIRTEIENSDSDYDREKLQERLAKLAGGVAVIKAGAATEVELKERKHRIEDAVRNAKAAVEEGIVAGGGVTLLQAAPALDKLKLTGDEATGANIVKVALEAPLKQIAFNSGMEPGVVAEKVRNLSVGHGLNAATGEYEDLLKAGVADPVKVTRSALQNAASIAGLFLTTEAVVADKPEKTAAPASDPTGGMVTSGHDAGSYVV

SEQ ID NO: 5 - DNA, 19 bp, synthetic construct
  source 1..19: mol_type = other DNA
gtggggggaggggcgttct

SEQ ID NO: 6 - DNA, 21 bp, synthetic construct
  source 1..21: mol_type = other DNA
attttacaccaacccccagtt